(19) United States
(12) Patent Application Publication
(10) Pub. No.: US 2023/0076397 A1
(43) Pub. Date: Mar. 9, 2023

(54) METHOD FOR SIMULTANEOUS CONVERSION OF HYDROGEN SOURCE AND A CARBON DIOXIDE SOURCE INTO FORMATE WITH IMPROVED YIELD

(71) Applicant: KOREA RESEARCH INSTITUTE OF CHEMICAL TECHNOLOGY, Daejeon (KR)

(72) Inventors: Young Kyu HWANG, Daejeon (KR); Changho YOO, Daejeon (KR); Dong Won HWANG, Daejeon (KR); Kyung-Ryul OH, Daejeon (KR); Do Young HONG, Daejeon (KR); Maeum LEE, Daejeon (KR)

(73) Assignee: KOREA RESEARCH INSTITUTE OF CHEMICAL TECHNOLOGY, Daejeon (KR)

(21) Appl. No.: 17/885,006

(22) Filed: Aug. 10, 2022

(30) Foreign Application Priority Data
Aug. 11, 2021 (KR) 10-2021-0105729

(51) Int. Cl. C07C 51/41

(52) U.S. Cl. CPC C07C 51/41

(57) ABSTRACT

Proposed is a high-yield simultaneous conversion method for a hydrogen source and a carbon dioxide source. The method significantly increases a yield of a formate through conversion of carbon dioxide. To this end, a carbon dioxide source and a hydrocarbon containing one or more hydroxy groups undergo a simultaneous conversion reaction in the presence of a solvent containing one or more alcohols and having a pH of 10 to 14.

### CROSS REFERENCE TO RELATED APPLICATION

The present application claims priority to Korean Patent Application No. 10-2021-0105729, filed Aug. 11, 2021, the entire contents of which is incorporated herein for all purposes by this reference.

### BACKGROUND OF THE INVENTION

### 1. Field of the Invention

The present disclosure relates to a method for simultaneous conversion of a hydrogen source and a carbon dioxide source for conversion of CO₂ to a formate with an improved yield. More particularly, the present disclosure relates to a method for simultaneous conversion of carbon dioxide and a hydrocarbon containing one or more hydroxy groups into a formate with an extremely improved yield by using a specific solvent.

### 2. Description of the Related Art

At present, with the industrial development, increased use of fossil fuels and the resulting surge in CO₂ emissions have been one of the big issues which should be solved. For this reason, there have been efforts to capture carbon dioxide and convert the captured carbon dioxide into useful chemicals.

CO₂ conversion has attracted attention in terms of preventing global warming by reducing CO₂ emissions and of solving the problem of resource depletion by recycling carbon resources. CO₂ conversion is valuable as a linking technology between renewable energy and the competitiveness improvement of biotechnology industry. Carbon dioxide is a low-energy compound requiring a lot of energy for conversion to useful resources. This is an obstacle to the commercial success of carbon dioxide conversion technologies. Therefore, if a catalyst capable of minimizing energy use and of improving reaction selectivity can be successfully developed, it is believed that the carbon dioxide conversion technology can be an extremely useful technology because the problematic carbon dioxide can be appropriately processed and value added useful materials can be recovered from carbon dioxide through a low-cost conversion process.

One such technique is a process of converting carbon dioxide to formic acid through a hydrogenation reaction, and the process can be represented by Reaction Formula 1. Formic acid is used in various industrial fields such as livestock feed processing, leather dyeing, and rubber synthesis. Since formic acid is low in combustibility and is easy to store and transport, the formic acid is widely used as a hydrogen reservoir.

(Reaction Formula 1)

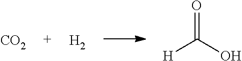

ΔG° aq (kcal/mol)=13.4

Although the hydrogenation reaction is widely used in the field of catalysts, since a hydrogen transfer reaction in which hydrocarbons including hydroxy groups, such as glycerol and glucose, are reacted with carbon dioxide so that hydrogen in the hydrocarbons can migrate into carbon dioxide is thermodynamically easier than a direct reaction in which hydrogen gas directly combines with carbon dioxide, research on the hydrogen transfer reaction has attracted attentions.

Particularly, glucose is known as a hydrogen source capable of being dehydrogenated by catalytic reaction at room temperature. A paper in the ChemSusChem journal (Hongfei Lin, Coupling Glulcose Dehydrogenation with CO₂ Hydrogenation by Hydrogen Transfer in Aqueous Media at Room Temperature, ChemSusChem, 1 Jun. 2018) discloses a technique for preparing formate by subjecting glucose as a hydrogen source and (NH₄)₂CO₃ as a carbon dioxide source to catalytic reactions at room temperature in an ethanol solvent. However, when (NH₄)₂CO₃ is used as a carbon dioxide source, it is difficult to source materials and ammonia by-products were produced during the reaction. For these problems, there was an attempt of using Na₂CO₃ that is easier in material supply and generates fewer by-products. However, the use of Na₂CO₃ caused a problem of significantly reducing the yield of formate production.

In consideration of these problems, the present disclosure is directed to providing a new method of producing a formate from carbon dioxide, in which the method can improve the conversion rate of carbon dioxide by improving the selectivity of the formate among products produced by dehydrogenation and hydrogenation reactions between sugars such as glucose serving as a hydrogen source and carbon dioxide.

On the other hand, PCT Patent Application Publication No. WO2020-018972 (2020.01.23) which is a conventional art relating to the present disclosure discloses a technique for producing formic acid by reacting an adduct of a hydrogen-added solvent, an amine or an amino acid serving as a carbon source, and CO₂ at 80° C. to 90° C. In addition, Korean Patent Application Publication No. 10-2020-0057644 (published as of 2020.05.26) discloses a technique for hydrogenating ammonium bicarbonate to produce formic acid. In addition, U.S. Patent Application Publication No. 2016-0137573 discloses a technique for hydrogenating carbon dioxide to produce formates. In the technique, carbon dioxide-derived compounds such as sodium bicarbonate (NaHCO₃) are hydrogenated in a non-uniform catalyst system including Pd and a carbon-based material.

Although these conventional art-related literatures disclose techniques of hydrogenating carbonate derived from carbon dioxide to produce formic acid or formate but do not disclose low-temperature high-yield formate production methods that use a metal carbonate or metal bicarbonate that does not contain ammonium as the carbon dioxide or carbon dioxide-derived carbonate.

### Related Art Literature

Patent Literature
(Patent Literature 1) PCT Patent Application Publication No. WO2020-018972 (published as of 2020.01.23)
(Patent Literature 2) Korean Patent Application Publication No. 10-2020-0057644 (published as of 2020.05.26)
(Patent Literature 3) U.S. Patent Application Publication No. 2016-0137573 (published as of 2016.05.19)

### Non-Patent Literature

(Non-Patent Literature 1) Hongfeil Lin, Coupling Glucose Dehydrogenation with CO₂ Hydrogenation by Hydrogen Transfer in Aqueous Media at Room Temperature, ChemSusChem, 1 Jun. 2018

### SUMMARY OF THE INVENTION

The present invention has been made to solve the problems occurring in the related art, and the present disclosure is directed to providing a method of producing a formate through simultaneous conversion of carbon dioxide and a hydrocarbon containing one or more hydroxy groups by using a catalytic reaction, the method being capable of producing the formate at a low temperature with a high yield while using a metal carbonate or a metal bicarbonate as a carbon dioxide source.

In order to solve the above problem, the present disclosure provides a method for simultaneous conversion of a hydrogen source and a carbon dioxide source. In the method, the hydrogen source is a hydrocarbon containing one or more hydroxy groups, and the carbon dioxide source is one or more materials selected from among carbon dioxide, metal carbonate, and metal bicarbonate. In addition, the hydrogen source and the carbon dioxide source are reacted in the presence of a solvent, in which the solvent is an aqueous solution containing one or more alcohols having 1 to 4 carbon atoms, and a solution in which the hydrogen source and the carbon dioxide source are dissolved is adjusted to have a pH within a range of 10 to 14.

In one embodiment of the present disclosure, the metal carbonate and the metal bicarbonate may be carbon dioxide derivatives formed by a reaction between carbon dioxide and a metal and/or a metal salt.

In one embodiment of the present disclosure, the simultaneous conversion may be carried out in the presence of a catalyst, and the catalyst may be made in a form in which one or more metals selected from among ruthenium (Ru), iridium (Ir), rhodium (Rh), platinum (Pt), palladium (Pd), and gold (Au) are supported on a support.

In another embodiment of the present disclosure, a basic material may be added to adjust the pH of the solution. The concentration of the carbon dioxide source in the solution may be in a range of 0.01M to 1M when the concentration is calculated on the basis of the amount of carbon dioxide, and the concentration ratio of the hydrogen source to the carbon dioxide source in the solution may be a range 0.1 to 10 in terms of the mole of hydrogen and the mole of carbon dioxide, respectively.

The metals in the metal carbonate and the metal bicarbonate may be K, Na, Li, Rb, or Cs. The content of alcohol in the aqueous solution may be in a range of 10% to 90% by weight. As the reaction conditions of the simultaneous conversion reaction, the reaction temperature may be in a range of 0° C. to 50° C., and the reaction pressure may be in a range of 1 to 50 bar. The content of alcohol in said aqueous solution is preferably in a range of 30% to 70% by weight and more preferably in a range of 40% to 60% by weight.

In addition, the present disclosure provides a method for simultaneous conversion of a hydrogen source and a carbon dioxide source, in which the hydrogen source is a hydrocarbon containing one or more hydroxy groups, the carbon dioxide source is a metal carbonate, the metal is one or more substances selected from among K, Rb, and Cs, and the co-converting is performed in an aqueous solution containing one substance selected from among ethanol, n-propanol, isopropanol, and t-butanol.

According to the present disclosure, the simultaneous conversion reaction of carbon dioxide and a saccharide uses a hydrocarbon containing one or more hydroxy groups as the hydrogen source and uses carbon dioxide, a metal carbonate, or a metal bicarbonate as the carbon dioxide source. Therefore, there is an advantage that by-products such as ammonia are not generated.

In addition, according to the present disclosure, since the aqueous solution containing an alcohol having 1 to 4 carbon atoms as the solvent, even with the use of a metal carbonate or metal bicarbonate as a carbon dioxide source, it is possible to produce a formate in a high yield.

### DESCRIPTION OF THE PREFERRED EMBODIMENTS

Hereinafter, preferred embodiments of the present disclosure that can be easily implemented by those skilled in the art will be described in detail. In describing the principles employed in the preferred embodiments of the present invention, well-known functions or constructions will not be described in detail when they may obscure the gist of the present invention.

The present disclosure relates to simultaneous conversion of carbon dioxide and a hydrogen source that is a hydrocarbon containing one or more hydroxy groups. One or more forms selected from among carbon dioxide molecules, a metal carbonate derived from carbon dioxide, and a metal bicarbonate derived from carbon dioxide may be introduced into the simultaneous conversion reaction as the carbon dioxide. The reaction according to the present disclosure improves the selectivity of formates among products obtained by the reaction.

As such, the embodiment of the present disclosure can convert sugars and carbon dioxide into formates with high yields by simultaneous conversion of sugars and carbon dioxide. Therefore, the present disclosure can be widely used in the field of carbon dioxide fixation, capture, conversion, or storage.

In another embodiment of the present disclosure, any hydrocarbons including one or more hydroxy groups can be used as the hydrogen source without particular limitations, but the hydrogen source will be preferably a sugar derived from biomass. More preferably, one or more sugars selected from among glucose, galactose, and lactose may be used as the hydrogen source, and most preferably, glucose may be used.

Hereinafter, a method for simultaneous conversion of carbon dioxide and a saccharide according to the present disclosure will be described in detail.

The simultaneous conversion reaction according to the present disclosure is carried out in the presence of a solvent, in which the solvent is an aqueous solution containing an alcohol having 1 to 4 carbon atoms. In this case, a catalyst may be used to promote the reaction. The catalyst may be a catalyst in which at least one type of noble metal is supported on a support as an active metal. The catalyst promotes a reaction between a hydrocarbon containing one or more hydroxy groups as a hydrogen source and one or more substances selected from among carbon dioxide, metal carbonates, and metal bicarbonates as a carbon dioxide source, and the reaction produces a formate.

In addition, as the active metal component in the catalyst, one or more noble metals selected from among ruthenium (Ru), iridium (Ir), rhodium (Rh), platinum (Pt), palladium (Pd), and gold (Au) may be used. Preferably, the noble metal may be platinum (Pt), rhodium (Rh), or palladium (Pd).

In addition, the support in the catalyst is a solid phase enabling a catalytic material to be dispersed and stably maintaining the dispersed catalytic material. The support is a carrier that holds the catalyst material in a highly dispersed manner so that the exposed surface area of the catalytic material can be maximized. To this end, the support usually refers to a material that is porous or has a large surface area and is mechanically, thermally, and chemically stable. The selection of the support is made depending on the diameter and volume of the pores in the support, the surface area, the strength, the chemical stability, and the shape. Since there are cases that the activity of the catalyst varies depending on the type of support, the support may be appropriately selected according to the type of active metal and the type of reaction.

Examples of the support according to the present disclosure include: activated carbon; carbon-phase materials such as graphite carbon; molecular sieves such as zeolite, metal-organic frameworks (MOF), etc.; ceramic materials such as hydrotalcite, perovskite, spinel (for example, CoAl₂O₄), etc.; metal oxides such as alumina, silica, etc.; sulfate-treated metal oxides such as ZrO₂—SO₄ or SnO₂—SO₄; and metal oxyhydroxides such as AlOOH, ZrO(OH)₂, CoOOH, etc. Preferably, the support may be made of activated carbon or graphite carbon.

The support may be purchased from among commercially available products or may be self-manufactured for use. For example, a carbon body formed as a support provided in a catalyst composite may be obtained by firing a metal-organic framework (MOF) used as a starting material.

In order to prepare the catalyst composite according to the present disclosure, the active metal may be supported using one of the methods known in the art. For example, impregnation, coprecipitation, solid phase crystallization, vapor deposition, washcoating, sol-gel, in-situ synthesis, etc., may be used.

In the most preferred embodiment, the catalyst comprises at least one of a carrier in which platinum (Pt) is supported on a carbon support and a carrier in which palladium (Pd) is supported on a carbon support. Preferably, the catalyst comprises a mixture of the carrier in which Pt is supported on a carbon support and the carrier in which Pd is supported on the carbon support.

In the carbon dioxide-derived metal carbonate and the carbon dioxide-derived metal bicarbonate, the metal may be sodium (Na), potassium (K), lithium (Li), rubidium (Rb), or cesium (Cs). Preferably, the metal is potassium (K).

In addition, in the simultaneous conversion method according to the disclosure, the content of alcohol included in the aqueous solution is in a range of 10% to 90% by weight. The content of alcohol is preferably in a range of 30% to 70% by weight and is more preferably in a range of 40% to 60% by weight.

In addition, according to the present disclosure, the pH of a solution containing a solvent, a carbon dioxide source, and a hydrogen source needs to be adjusted to be within a range of 10 to 14 and preferably within a range of 11 to 13. The yield of a formate is lowered when the pH of the solution is outside the range of 10 to 14.

The pH of the solution may be adjusted by adding a basic material to the solution. Non-limiting examples of the basic material include one or more materials selected from among metal hydroxides such as KOH, NaOH, Ca(OH)₂, metal alkoxides, and amines.

The concentration of the carbon dioxide source in the solution may be in a range of 0.01 to 1 M in terms of the concentration of carbon dioxide. The molar ratio of the hydrogen source to the carbon dioxide source may be in a range of 0.1 to 10 when the moles of the hydrogen source and the carbon dioxide source are converted into the moles of hydrogen and carbon dioxide, respectively.

In the simultaneous conversion reaction of a carbon dioxide source and a hydrocarbon containing one or more hydroxy groups, the reaction temperature is preferably in a range of 0° C. to 50° C., and the reaction pressure is preferably in a range of 1 to 50 bar.

In addition, preferably, the reactants participating in the reaction include 0.1 to 10 parts by weight of the catalyst per 100 parts by weight of the solvent.

In the simultaneous conversion reaction of a carbon dioxide source and a saccharide, the simultaneous conversion reaction may further include a step of converting carbon dioxide into a metal carbonate and/or a metal bicarbonate by reacting a metal, a metal salt, or both with carbon dioxide before reacting the hydrogen source with the carbon dioxide source.

Hereinafter, the effects of the present disclosure will be described in more detail with reference to examples.

### Experimental Examples 1 to 5

0.2162 g of glucose (Sigma Aldrich, G8270) as a hydrogen source and 0.0829 g of K₂CO₃ (Sigma Aldrich, 209619) as a carbon dioxide source were added to an aqueous solution of 2.651 g of alcohol and 2.651 g of distilled water as described in Table 1 below to prepare solutions containing 0.2 M of glucose and 0.1 M of K₂CO₃. Next, the pH of each of the solutions was measured. In Experimental Examples 1 to 5, the pH of each of the solutions was about 11.8. Next, 0.02 g of Pt/C (Sigma Aldrich, 205931) in which Pt is contained in an amount of 5% by weight, 0.08 g of Pd/C (Sigma Aldrich, 205680) in which Pd is contained in an amount of 5% by weight, and a magnetic bar were added to each of the solution, and the resulting solutions were stirred at 25° C. for 24 hours to obtain reaction products. The resulting samples were analyzed using HPLC (Shodex Sugar SH1101) and the results are shown in Table 1 below. Cony. in Table 1 below represents the conversion rate of glucose, and the numerical values of Gluconate (%) and the like represents yields calculated as follows:

Glucose Cony.={(initial glucose mole number)−(post-reaction glucose mole number)/(initial glucose mole number)}×100(%)

Gluconate Yield={(number of moles of gluconate produced)/(number of moles of glucose initially added)}×100(%)

Sorbitol Yield{(number of sorbitol moles produced)/(number of moles of glucose initially added)}×100(%)

Formic Acid(FA)Yield={(number of moles of FA produced)/(number of moles of CO₂)}×100(%)

TABLE 1

| | | | Yield (%) | | |
|---|---|---|---|---|---|
| Classification | Solvent | Conv.(%) | Gluconate | Sorbitol | FA |
| Experimental | H₂O | 48.0 | 35.6 | 5.8 | 2.3 |
| Example 1 | | | | | |
| Experimental | 50% by | 53.6 | 40.6 | 5.7 | 6.1 |
| Example 2 | weight of | | | | |
| | MeOH | | | | |
| Experimental | 50% by | 54.6 | 44.3 | 6.6 | 23.2 |
| Example 3 | weight of | | | | |
| | EtOH | | | | |
| Experimental | 50% by | 53.2 | 40.2 | 7.4 | 15.2 |
| Example 4 | weight of | | | | |
| | 1-propanol | | | | |
| Experimental | 50% by | 53.8 | 42.5 | 6.3 | 30.5 |
| Example 5 | weight of | | | | |
| | IPA | | | | |
| Experimental | 50% t-BuOH | 49.1 | 43.5 | 5.1 | 18.3 |
| Example 6 | | | | | |

The results of simultaneous conversion using glucose and K₂CO₃ are shown in Table 1. The yield of formic acid increased when a 50% aqueous alcohol solution was used as the solvent compared to the case where water was used as the solvent. When 50% IPA was used as the solvent, the yield was best (i.e., 30.5%).

### Experimental Examples 7 to 12

Except for using 0.0636 g of Na₂CO₃ (Sigma Aldrich, 223530) as the carbon dioxide source, the experiments were performed in the same manner as in Examples 1 to 6, respectively, and the results are shown in Table 2 below. In Experimental Examples 7 to 12, the pH of each of the solutions was about 11.5.

TABLE 2

| | | | Yield (%) | | |
|---|---|---|---|---|---|
| Classification | Solvent | Conv.(%) | Gluconate | Sorbitol | FA |
| Experimental | H₂O | 48.0 | 35.6 | 5.8 | 0.5 |
| Example 7 | | | | | |
| Experimental | 50% by | 55.8 | 23.4 | 20.9 | 2.0 |
| Example 8 | weight of | | | | |
| | MeOH | | | | |
| Experimental | 50% by | 50.3 | 38.6 | 5.7 | 4.7 |
| Example 9 | weight of | | | | |
| | EtOH | | | | |
| Experimental | 50% by | 42.0 | 31.3 | 3.1 | 2.6 |
| Example 10 | weight of | | | | |
| | 1-propanol | | | | |
| Experimental | 50% by | 49.8 | 38.4 | 5.8 | 14.9 |
| Example 11 | weight of | | | | |
| | IPA | | | | |
| Experimental | 50% t-BuOH | 39.6 | 34.5 | 4.8 | 13.3 |
| Example 12 | | | | | |

Table 2 shows the results of using Na₂CO₃ as the carbon dioxide source instead of K₂CO₃. The yield of formic acid increased when using a 50% alcohol aqueous solution was used as the solvent as in Experiment Examples 1 to 5, but the increase in this case was lower than the in the case of using K₂CO₃.

### Experimental Examples 13 to 19

Experimental Examples 13 to 19 used carbon dioxide sources other than K₂CO₃ and Na₂CO₃ which were used as the carbon dioxide sources in Experimental Examples 1 to 12.

Experimental Examples 13 to 19 used Rb₂CO₃, Cs₂CO₃, (NH₄)₂CO₃, NaHCO₃, KHCO₃, NH₄HCO₃, and KHCO₃, respectively, in an amount of 0.1 M. Experimental Example 19 was performed in the same manner as in Experimental Example 1 except that 0.1 M of KOH was used and an aqueous solution of 50% by weight of water and 50% by weight of isopropanol was used as the solvent.

TABLE 3

| | CO₂ | | Conv. | Yield (%) | | |
|---|---|---|---|---|---|---|
| Classification | source | pH | (%) | Gluconate | Sorbitol | FA |
| Experimental | R₂CO₃ | 11.7 | 52.8 | 45.5 | 5.8 | 29.8 |
| Example 13 | | | | | | |
| Experimental | CS₂CO₃ | 11.8 | 55.8 | 43.4 | 5.9 | 26.7 |
| Example 14 | | | | | | |
| Experimental | (NH₄)₂CO₃ | 8.8 | 30.3 | 16.3 | 2.5 | 0.5 |
| Example 15 | | | | | | |
| Experimental | NaHCO₃ | 8.0 | 18.2 | 14.0 | 4.1 | 0.4 |
| Example 16 | | | | | | |
| Experimental | KHCO₃ | 8.0 | 22.5 | 14.4 | 7.8 | 0.7 |
| Example 17 | | | | | | |
| Experimental | NH₄HCO₃ | 7.8 | 23.2 | 16.9 | 2.1 | 0.4 |
| Example 18 | | | | | | |
| Experimental | KHCO₃ + | 12.0 | 47.6 | 35.2 | 4.2 | 24.3 |
| Example 19 | KOH | | | | | |

Referring to Table 3, the yield of formic acid was high to be about 30% when Rb₂CO₃ or Cs₂CO₃ was used. When (NH)₄CO₃, NaHCO₃, KHCO₃, or NH₄HCO₃ was used, the pH of the solution was low, resulting in that the glucose conversion rate and the yield of all products were extremely low.

When KOH was added to KHCO₃ to adjust the pH of the solution to fall within the range presented by the present disclosure, the glucose conversion rate and the yield of all products were increased. From this result, it is confirmed that the pH of the solution during the reaction as well as the type of solvent is a principal factor.

### Experimental Examples 20 to 24

Experimental Examples 20 to 24 were performed to check change in the yield with changing concentrations of alcohol. The total volume of the solvent was fixed to be the same as in Experimental Example 1, and an aqueous solution of water and isopropanol was used as the solvent. The results of the experiments were obtained by varying the weight ratio of isopropanol in the aqueous solution. (Experimental Example 1: 6 g of water, Experimental Example 20: 5.257 g of water and 0.584 g IPA, Experimental Example 21: 3.912 g of water and 1.677 g of IPA, Experimental Example 5: 2.641 g of water and 2.641 g of IPA, Experimental Example 22: 1.536 g of water and 3.585 g of IPA, Experimental Example 23: 0.482 g of water and 4.337 g of IPA, Experimental Example 24: 4.716 g of IPA). K₂CO₃ was used as the carbon dioxide source. In Experimental Examples 20 to 24, the pH of each of the solutions was about 11.8.

TABLE 4

| | | Conv. | Yield (%) | | |
|---|---|---|---|---|---|
| Classification | Solvent | (%) | Gluconate | Sorbitol | FA |
| Experimental | 0% by | 48.0 | 35.6 | 5.8 | 2.3 |
| Example 1 | weight | | | | |
| | of IPA | | | | |
| Experimental | 10% by | 45.9 | 35.4 | 3.8 | 9.2 |
| Example 20 | weight | | | | |
| | of IPA | | | | |
| Experimental | 30% by | 51.5 | 41.0 | 5.0 | 18.0 |
| Example 21 | weight | | | | |
| | of IPA | | | | |
| Experimental | 50% by | 53.8 | 42.5 | 6.3 | 30.5 |
| Example 5 | weight | | | | |
| | of IPA | | | | |
| Experimental | 70% by | 46.9 | 32.8 | 4.7 | 18.2 |
| Example 22 | weight | | | | |
| | of IPA | | | | |
| Experimental | 90% by | 51.8 | 21.4 | 5.1 | 4.2 |
| Example 23 | weight | | | | |
| | of IPA | | | | |
| Experimental | 100% by | 22.2 | 8.7 | 2.2 | 1.0 |
| Example 24 | weight | | | | |
| | of IPA | | | | |

Referring to the results of Table 4, the yield of FA changed according to the IPA content. The highest yield was obtained at 50% by weight of IPA. When the IPA content was reduced or increased than 50% by weight, the yield of FA decreased.

### Experimental Examples 25 to 28

Experimental examples 25 to 28 were measurements of changes in the yield of FA according to the concentration of K₂CO₃ and the concentration of glucose. The solvent was an alcohol aqueous solution containing 50% by weight of water and 50% by weight of IPA. The total weight (g) of the solvent was fixed to be the same as in Experiment Example 1, and the concentration of K₂CO₃ and the concentration of glucose in the alcohol aqueous solution were changed as shown in Table 5 below.

TABLE 5

| | | | | Conv. | Yield (%) | | |
|---|---|---|---|---|---|---|---|
| Classification | Glucose | K₂CO₃ | pH | (%) | Gluconate | Sorbitol | FA |
| Experimental | 0.2 M | 0.05 M | 11.6 | 47.0 | 41.9 | 2.9 | 1.2 |
| Example 25 | | | | | | | |
| Experimental | 0.2 M | 0.2 M | 11.9 | 80.5 | 59.7 | 21.3 | 24.0 |
| Example 26 | | | | | | | |
| Experimental | 0.4 M | 0.2 M | 11.9 | 58.5 | 35.3 | 22.3 | 20.3 |
| Example 27 | | | | | | | |
| Experimental | 1 M | 0.5 M | 12.1 | 51.4 | 17.1 | 32.4 | 25.7 |
| Example 28 | | | | | | | |

Referring to Table 5, when the concentration of K₂CO 3 was low, the pH was low and the ability to receive hydrogen was low, resulting in a low yield of formic acid. When the concentration of K₂CO₃ was high, the pH was high, resulting in a high yield of formic acid.

### Experimental Examples 29 to 34

Experimental Examples 29 to 34 were performed in the same manner as in Experimental Examples 1 to 6 except that galactose or lactose was used as the hydrogen source. The results are shown in Table 6. In Experimental Examples 29 to 34, the pH of each of the solutions was about 11.8.

TABLE 6

| | | | Yield (%) | | |
|---|---|---|---|---|---|
| Classification | Solvent | Conv.(%) | Galactonate | Dulcitol | FA |
| Experimental | H₂O | 48.6 | 33.4 | 7.3 | 2.1 |
| Example 29 | | | | | |
| Experimental | 50% by | 50.2 | 34.9 | 9.0 | 10.9 |
| Example 30 | weight of | | | | |
| | MeOH | | | | |
| Experimental | 50% by | 54.9 | 37.4 | 10.5 | 20.8 |
| Example 31 | weight of | | | | |
| | EtOH | | | | |
| Experimental | 50% by | 52.0 | 37.8 | 10.4 | 17.7 |
| Example 32 | weight of | | | | |
| | 1-propanol | | | | |
| Experimental | 50% by | 55.9 | 39.5 | 12.0 | 22.5 |
| Example 33 | weight of | | | | |
| | IPA | | | | |
| Experimental | 50% by | 50.0 | 36.0 | 10.3 | 14.6 |
| Example 34 | weight of | | | | |
| | t-BuOH | | | | |

Referring to Table 6, the yield of formic acid was higher in a 50% alcohol solution than in water, as was the case with glucose, and similar tendencies were observed depending on the type of alcohol.

### Experimental Examples 35 to 40

Experimental Examples 35 to 40 were performed in the same manner as in Experimental Examples 1 to 6 except that lactose was used as the hydrogen source. The results are shown in Table 7. In Experimental Examples 35 to 40, the pH of each of the solutions was about 11.8.

TABLE 7

| | | | Yield (%) | | |
|---|---|---|---|---|---|
| Classification | Solvent | Conv.(%) | Lactobionate | Lactitol | FA |
| Experimental | H₂O | 43.0 | 41.9 | 0.1 | 2.0 |
| Example 35 | | | | | |
| Experimental | 50% by | 47.2 | 46.7 | 0.0 | 7.3 |
| Example 36 | weight | | | | |
| | of MeOH | | | | |
| Experimental | 50% by | 64.9 | 44.5 | 4.5 | 17.5 |
| Example 37 | weight | | | | |
| | of EtOH | | | | |
| Experimental | 50% by | 41.5 | 33.8 | 5.3 | 17.6 |
| Example 38 | weight of | | | | |
| | 1-propanol | | | | |
| Experimental | 50% by | 45.8 | 35.7 | 6.1 | 20.4 |
| Example 39 | weight | | | | |
| | of IPA | | | | |
| Experimental | 50% by | 41.2 | 37.8 | 3.3 | 17.6 |
| Example 40 | weight of | | | | |
| | t-BuOH | | | | |

Referring to Table 7, even when lactose was used as the hydrogen source, as was the case with glucose or galactose, the yield of formic acid was higher in a 50% alcohol aqueous solution than in water, and similar tendencies were observed depending on the type of alcohol.

While the present disclosure has been described with reference to examples presented above, those skilled in the art will appreciate that the examples are presented only for illustrative purposes. On the contrary, it will be understood that various modifications and equivalents to the examples are possible. Accordingly, the technical scope of the present disclosure should be defined by the following claims.

What is claimed is:

1. A method for simultaneous conversion of a hydrogen source and a carbon dioxide source into a formate with an improved yield, the method using a simultaneous conversion reaction of the hydrogen source and the carbon dioxide source, wherein the hydrogen source is a hydrocarbon containing one or more hydroxy groups,
the carbon dioxide source is one or more materials selected from among carbon dioxide molecules, metal carbonates, and metal bicarbonates,
the simultaneous conversion reaction is performed in an aqueous solution containing one or more alcohols having 1 to 4 carbon atoms, and
the solution in which the hydrogen source and the carbon dioxide source are dissolved is adjusted to have a pH within a range of 10 to 14.

2. The method of claim 1, wherein the metal carbonate and the metal bicarbonate are carbon dioxide derivatives formed by a reaction between carbon dioxide and a metal and/or metal salt.

3. The method of claim 1, wherein the simultaneous conversion is performed in the presence of a catalyst, and the catalyst has a form in which one or more metals selected from among ruthenium (Ru), iridium (Ir), rhodium (Rh), platinum (Pt), palladium (Pd), and gold (Au) are supported on a support.

4. The method of claim 1, wherein a basic material is further added to adjust the pH of the solution.

5. The method of claim 1, wherein the carbon dioxide source has a concentration of 0.01 M to 1 M in the solution.

6. The method of claim 1, wherein the hydrogen source and the carbon dioxide source are mixed in a ratio in a range of 0.1 to 10 in the solution in terms of moles of hydrogen and carbon dioxide.

7. The method of claim 1, wherein the metal in the metal carbonate salt and the metal in the metal bicarbonate salt are K, Na, Li, Rb, or Cs.

8. The method of claim 1, wherein the one or more alcohols in the aqueous solution have a concentration within a range of 10% to 90% by weight.

9. The method of claim 1, wherein the reaction is performed at a temperature in a range of 0° C. to 50° C. and at a pressure in a range of 1 to 50 bar.

10. A method for simultaneous conversion of a hydrogen source and a carbon dioxide source, the method using a simultaneous conversion reaction of the hydrogen source and the carbon dioxide source, wherein the hydrogen source is a hydrocarbon containing at least one hydroxyl group,
the carbon dioxide source is a metal carbonate,
the metal is at least one of K, Rb, and Cs, and
the simultaneous conversion reaction is performed in an aqueous solution containing at least one of ethanol, n-propanol, isopropanol, t-butanol.
